(19) United States
(12) Patent Application Publication
(10) Pub. No.: US 2017/0276653 A1
(43) Pub. Date: Sep. 28, 2017

(54) METABOLIC BIOMARKERS FOR DIFFERENTIAL DIAGNOSIS OF STABLE ANGINA PECTORIS AND ACUTE CORONARY SYNDROME

(71) Applicant: Lianwen QI, Nanjing (CN)

(72) Inventors: Lianwen QI, Nanjing (CN); Yong FAN, Nanjing (CN); Ping LI, Nanjing (CN); Wei ZHU, Nanjing (CN); Yan CHEN, Nanjing (CN)

(73) Assignee: Lianwen QI, Nanjing (CN)

(21) Appl. No.: 15/273,728

(22) Filed: Sep. 23, 2016

(30) Foreign Application Priority Data
Mar. 24, 2016 (CN) 201610171544.3

(51) Int. Cl. G01N 30/72; G01N 33/49

(52) U.S. Cl. CPC G01N 30/7206; G01N 30/7233; G01N 33/492; G01N 2570/00; G01N 2800/324; G01N 2560/00

(57) ABSTRACT

A panel of metabolic biomarkers for differential diagnosis of stable angina pectoris and acute coronary syndrome is published herein. The panel comprises one or more of the metabolic biomarkers, including malic acid, taurine, arachidonic acid, citramalic acid, methionine, pentadecanoic acid. Single use of the 6 differential metabolites provided clinically diagnostic value of SA vs. ACS with AUC>0.7. When combined, the more metabolites, the larger of AUC. The highest AUC of 0.987 was obtained when all of the six metabolites were combined to distinguish SA vs. ACS with sensitivity 96.8% and specificity 97.7% using optimal cut-off value. The metabolic biomarkers provided by the invention can be used for differential diagnosis of SA vs. ACS of high accuracy, sensitivity and specificity.

### TECHNICAL FIELD

The present invention is of biochemistry technology, involving metabolic biomarkers for characterizing the various types of coronary artery disease, especially a panel of metabolic biomarkers for differential diagnosis of stable angina pectoris and acute coronary syndrome.

### BACKGROUND

Coronary artery disease (CAD), also named ischemic heart disease, involves the atherosclerosis of artery supplying heart muscle, which is caused by vascular stenosis or plaque rupture and occlusion accompanying myocardial ischemia, hypoxia or necrosis. The disease can led to a series of serious cardiovascular problems such as angina and myocardial infarction. CAD remains one of the main killers to human health with high morbidity, high disability rate, high recurrence rate, high mortality, and multiple comorbidities and thus it has been one of the main diseases threatening people's health in China.

Based on pathophysiologic mechanisms, CAD is currently divided into stable coronary artery disease (i.e., stable angina pectoris, SA) and acute coronary syndrome (ACS). ACS is further divided into unstable angina pectoris (UA) and acute myocardial infarction (AMI). The emergence and progression of CAD are as follows: normal coronary artery (NCA), coronary atherosclerosis (CA), SA, UA, and AMI.

Coronary artery atherosclerosis is the main cause and early stage of CAD. Coronary atherosclerosis is a common progressive arterial disease. The lesions are mainly involved in the medium sized muscular arteries with arterial intimal lipid deposition and proliferation of smooth muscle cells, which can lead to the formation of local plaque and make the arteries hard. When the plaque ruptures, thrombosis, embolism and hemorrhage happen and lead to partial or complete occlusion of the involved arteries. They are seen as the complications of atherosclerosis clinically. Early stage of coronary artery atherosclerosis may emerge before 10 years old while it takes 20 to 30 years to form artery stenosis. Because of non-obvious clinical symptoms of atherosclerosis in early stage, it is not easy to be noticed or regarded. Therefore, the early prevention and diagnosis of coronary artery atherosclerosis can effectively prevent the occurrence of CAD.

Coronary angiography can accurately determine the degree of stenosis of coronary artery and it is the “gold standard” for diagnosis of CAD (research on the differences in clinical diagnosis of coronary heart disease by using coronary angiography gold standards and conventional diagnosis techniques, Shu Rongwen, et. al., Journal of Navy Medicine, 2015, 4). Unfortunately, the invasive coronary angiography based on intervention surgery is costly and can simply determine the degree of stenosis of coronary artery. Moreover, Doctors need to refer to the patients' electrocardiogram, echocardiography, treadmill exercise test, CT and other test results to make the final diagnosis, which may cause erroneous diagnosis or missed diagnosis because of subjective judgment of doctors or patients' unclear statement. This affects the prognosis of the patients a lot. To reduce the threat to patients' life and improve their life quality, a cheap, non-invasive and simple diagnostic method with high diagnostic accuracy is urgently needed.

Metabolomics as an important part of system biology, focuses on endogenous metabolites in organism and their changes with internal and external factors. It can analyze body fluids quickly and non-invasively such as blood and urine, and obtain the metabolites that indicate the various biochemical reactions from the differences in metabolic profiles. The commonly used analytical techniques currently include nuclear magnetic resonance (NMR), mass spectrometry (LC-MS/GC-MS) and so on. Because of the simplicity in sample preparation, high sensitivity and wide linear range, LC-MS/GC-MS is becoming a more and more commonly used technology in metabolomics investigations. Plasma analysis is a common diagnostic method in clinical and is widely used because of its simplicity, low cost, and relatively non-invasiveness.

Up to now, no studies on plasma metabolomic profiling for characterizing different types of CAD were reported. Plasma metabolomic profiling of people with normal coronary artery and patients with coronary artery atherosclerosis and various types of CAD is of high meaning for clinical diagnosis of CAD and differentiation of the various types of CAD in early stage.

### SUMMARY OF THE INVENTION

Firstly, the invention aims to afford a panel of metabolic biomarkers for differential diagnosis of stable angina pectoris and acute coronary syndrome. The metabolic biomarkers coexisting in plasma can be analyzed simultaneously. Secondly, the invention aims to afford a method for sensitive analysis of the metabolic biomarkers. Thirdly, the invention aims to afford a detection kit of the metabolic biomarkers for differential diagnosis of stable angina pectoris and acute coronary syndrome, making the diagnosis more convenient and standardized.

The above aims are accomplished by the following technologies.

A panel of metabolic biomarkers for differential diagnosis of stable angina pectoris and acute coronary syndrome comprises one or more of the metabolic biomarkers, including malic acid, taurine, arachidonic acid, citramalic acid, methionine, pentadecanoic acid.

As an optimized technology, the panel of metabolic biomarkers for differential diagnosis of stable angina pectoris and acute coronary syndrome comprises any two of the metabolic biomarkers.

As an optimized technology, the panel of metabolic biomarkers for differential diagnosis of stable angina pectoris and acute coronary syndrome comprises any three of the metabolic biomarkers.

As an optimized technology, the panel of metabolic biomarkers for differential diagnosis of stable angina pectoris and acute coronary syndrome comprises any four of the metabolic biomarkers.

As an optimized technology, the panel of metabolic biomarkers for differential diagnosis of stable angina pectoris and acute coronary syndrome comprises any five of the metabolic biomarkers.

As an optimized technology, the panel of metabolic biomarkers for differential diagnosis of stable angina pectoris and acute coronary syndrome comprises all of the six metabolic biomarkers.

As an optimized technology, all the metabolic biomarkers are defined as plasma metabolic biomarkers.

Methods for qualitative or quantitative analysis of the panel of metabolic biomarkers for differential diagnosis of stable angina pectoris and acute coronary syndrome comprise LC-MS and/or GC-MS. LC-MS and GC-MS show low detection limit and high sensitivity and thus can analyze metabolic biomarkers in biosamples sensitively.

A detection kit for differential diagnosis of stable angina pectoris and acute coronary syndrome comprises the reference standards of the panel of metabolic biomarkers. The reference standards are individually packaged or packaged together. The detection kit can promote the diagnostic standardization and thus improve the convenience and reproducibility of the diagnostic method.

As an optimized technology, the kit comprises the solvents for dissolving the reference standards and/or for extraction and enrichment of the panel of metabolic biomarkers.

The advantages of this invention:

Firstly, the metabolic biomarkers provided by the invention can accurately distinguish stable angina pectoris from acute coronary syndrome. According to the receiver operating characteristic (ROC) analysis, better prediction accuracy is obtained with AUC closer to 1.0 in the case of AUC higher than 0.5. AUC between 0.5˜0.7 means lower diagnostic accuracy; AUC between 0.7˜0.9 means certain diagnostic accuracy; AUC higher than 0.9 means high diagnostic accuracy. It is proved that single one of the metabolic biomarkers published by the invention offered AUC larger than 0.7 while combinations of the metabolic biomarkers offered AUC closer to 1 with better diagnostic accuracy. Combinations of all the six metabolic biomarkers offered AUC closest to 1 with the best diagnostic accuracy.

Secondly, the analytical method provided in the invention for the metabolic biomarkers is sensitive and accurate with robust reliability.

Thirdly, the detection kit is capable of differential diagnosis of SA and ACS and it can improve the diagnostic convenience and promote the diagnostic standardization.

### DETAILED DESCRIPTION OF THE EMBODIMENTS

Several detailed cases for the applications of this invention are provided below. If not clarified, all the instruments and reagents are conventional, and all the operations used are public to a person skilled in the art.

### Embodiment 1

### Plasma Metabolomic Profiling of SA Patients and ACS Patients

Part One: Samples and Methods

1. Plasma Samples

Peripheral venous blood plasma of 280 SA patients, 320 ACS patients and 350 NCA enrolled from Jiangsu province hospital between September 2010 and June 2015 was collected. All the patients had signed the patient informed consent. All the SA patients, ACS patients and NCA were confirmed by coronary angiography. The ages and genders of NCA matched with those of SA and ACS patients. All the included patients had normal heart, lung, liver, kidney function and normal hematopoiesis.

Fasting blood was collected.

2. Reagents

Acetonitrile and formic acid (UPLC grade) were purchased from ROE company (USA). Methanol and chloroform (HPLC grade) were purchased from Jiangsu hanbon Science & Technology. Chlorinated methoxyamine and N-methyl-N-(trimethylsilyl)trifluoroacetamide (containing 1% trimethyl chlorosilane) were purchased from Sigma-Aldrich (USA). Deionized water was prepared by Milli-Q ultra pure water system (Millipore, USA). The reference standards of malic acid, taurine, arachidonic acid, citramalic acid, methionine and pentadecanoic acid were purchased from Sigma-Aldrich (USA).

3. Plasma Metabolomic Profiling

3.1. Profiling by UPLC-Q/TOF-MS

3.1.1. Sample Preparation

Response surface method (RSM) was applied to optimize the extraction solvent. The extraction and enrichment efficiency of metabolites in plasma with acetonitrile, methanol, ethanol, chloroform and water was investigated to get more peak numbers and larger total peak area in both ESI+ and ESI− modes. Data were evaluated by multivariate analysis, and variable importance to projection (VIP value) in PLS model was applied to reflect the importance of variables to model response. The VIP values of acetonitrile, methanol, ethanol, chloroform, water were 1.503, 0.802, 0.651, 0.688, 0.987 respectively and acetonitrile got the best extraction efficiency. So acetonitrile was chosen as extration solvent to extract plasma samples.

100 μL plasma was added to a 1.5 mL centrifuge tube, 400 μL acetonitrile was added, mixed 30 seconds by vortex, centrifugated for 10 minutes at 13,000 r/min at 4° C. Then, 200 μL supernatant was transferred to another 1.5 mL centrifuge tube, dried the liquid with nitrogen at room temperature and 300 μL 20% acetonitrile water solution was used to dissolve the residue.

3.1.2. Experimental Parameters

Parameters of UPLC-Q/TOF-MS

Liquid chromatographic separation for processed plasma was achieved on a 100×2.1 mm Waters BEH C18 column (particle size 1.7 μm) using a 1290 Infinity System (USA). Column temperature was 25° C. Temperature of injection chamber was room temperature. Injection volume was 2 μL. Mobile phase A was 0.1% formic acid-water solution (V/V) and mobile phase B was 0.1% formic acid-acetonitrile solution (V/V) in both ESP+ and ESI− modes. Linear gradient elution condition: 0˜1 min, 0˜30% mobile phase B; 1˜3 min, 30˜60% mobile phase B; 3˜8 min, 60˜90% mobile phase B; 8˜9 min, 90˜100% mobile phase B; 9˜10 min, 100% mobile phase B. Flow rate was 0.3 mL/min. The eluent was totally directed to the mass spectrometry detector.

Mass spectrometry was performed on a 6530 Quadrupole-Time of Flight system (all devices from Agilent Technologies, USA). Detection mode, ESI+ and ESI−; flow rate of drying gas, 7 L/min; temperature of drying gas, 300° C.; ionization temperature, 100° C.; capillary voltage, 3000V in both ESI+ and ESI− modes; collision voltage, 100V. Dry gas and cone gas were high purity nitrogen gas. Data were acquired three times per second in full scan mode. Scan mass range was set between m/z 100-1000 Da.

3.2. Profiling by GC-Q/MS

3.2.1. Sample Preparation

200 μL plasma was added to a 1.5 mL centrifuge tube, 50 μL citramalic acid solution (1 mg/mL) was added as Internal standard, then 400 μL mixed solution of methanol, chloroform and water (2.5:1:1, V/V/V) was added, then shook 30 minutes at 1200 r/min incubated in constant temperature (70° C.) metal bath, centrifugated for 5 minutes at 16,000 g at 4° C. Then, 500 μL supernatant was transferred to another 1.5 mL centrifuge tube, added 500 μL distilled water, mixed by vortex. centrifugated for 5 minutes at 16,000 g at 4° C., 500 μL supernatant was transferred to another 1.5 mL centrifuge tube, dried the liquid with nitrogen at room temperature, 80 μL methoxamine pyridine solution was used to dissolve the residue and oximated 8 hours at 50° C. After that, added 60 μL N-methyl-N-(trimethylsilyl)trifluoroacetamide and reacted 2 hours at 70° C.

3.2.2 Experimental Parameters

Parameters of GC-Q/MS. GC-Q/MS was performed on Agilent 7890B-5977A gas chromatography/mass spectrometer (GC/MS, USA). Capillary column: HP-5MS (30.0 m×0.25 mm, thickness 0.25 μm). Carrier gas was high purity helium and its flow rate was 1.0 mL/min. Injection volume was 2 μL. By temperature programming, the initial oven temperature was 80° C., then increased to 300° C. at a rate of 5° C./min and held for 6 min. Splitless injection, the injector temperature, 300° C.; interface temperature, 300° C.; source temperature, 200° C.; electron energy, 50 eV; solvent delay, 3 min. The MS detector was performed in full-scan reaction monitoring mode and recorded across the range m/z 30-600 Da.

4. Data Processing and Analysis

The acquired MS data from UPLC-Q/TOF-MS and GC-Q/MS were exported to SIMCA software (version 13.0.2, Umetrics). The OPLS-DA (orthogonal partial least squares—discriminant analysis) model was established to search for the metabolites that contributed a lot to the metabolic profiles between SA patients and ACS patients with VIP value higher than 1.0 and p value lower than 0.01.

The structures of the differential metabolites were preliminarily identified based on exact molecular weights in HMDB (http://www.hmdb.ca/) and Metline (http://metlin.scripps.edu/) databases and MS/MS spectra from mass spectroscopy. The structures were confirmed with the molecular weights, chromatographic retention times and multi stage MS spectra of the purchased reference standards.

Part Two: Results

Six differential metabolites were screen out including malic acid, taurine, arachidonic acid, citramalic acid, methionine, pentadecanoic acid.

Compared with SA Patients, the six differential metabolites with up-regulated or down-regulated trend were identified in the plasma of ACS Patients. It was proved that malic acid and citramalic acid down-regulated 0.7˜0.8 times while taurine, arachidonic acid, methionine and pentadecanoic acid up-regulated 0.7˜0.8 times in plasma of ACS patients compared with those of SA patients by quantitative analysis with reference standards. Thus it can be seen, the levels of the above six differential metabolites were significantly different between SA Patients and ACS Patients. The six differential metabolites can be used as metabolic biomarkers for differential diagnosis of SA and ACS.

### Embodiment 2

### Validation of the Diagnostic Ability by Using the Six Differential Metabolites to Distinguish SA from ACS by ROC Analysis

Receiver operating curve (ROC) analysis was applied to validate the possibility of using the six differential metabolites to distinguish SA from ACS ACS. Single use of malic acid, taurine, arachidonic acid, citramalic acid, methionine, pentadecanoic acid provided clinically diagnostic value of SA vs. ACS with AUC>0.7. When combined, the more metabolites, the larger of AUC. The highest AUC of 0.987 was obtained when all of the six metabolites were combined to distinguish SA vs. ACS with sensitivity 96.8% and specificity 97.7% using optimal cut-off value. The AUC, sensitivity and specificity of ROC analysis with single metabolite or combination of any two to five metabolites were listed in table 1 to 3.

TABLE 1
Single use of the 6 differential metabolites
to distinguish SA from ACS

| Single metabolite | AUC | Sensitivity | Specificity |
|---|---|---|---|
| Malic acid | 0.881 | 88.0% | 89.2% |
| Taurine | 0.868 | 84.7% | 85.9% |
| Arachidonic acid | 0.846 | 82.5% | 83.7% |
| Citramalic acid | 0.809 | 78.8% | 80.0% |
| Methionine | 0.793 | 77.2% | 78.4% |
| Pentadecanoic acid | 0.772 | 75.1% | 76.3% |

TABLE 2
Combination of any two of the 6 differential
metabolites to distinguish SA from ACS

| Combined metabolites | | AUC | Sensitivity | Specificity |
|---|---|---|---|---|
| Malic acid | Taurine | 0.916 | 92.6% | 93.3% |
| | Arachidonic acid | 0.909 | 91.0% | 92.9% |
| | Citramalic acid | 0.905 | 90.3% | 91.1% |
| | Methionine | 0.899 | 89.8% | 90.6% |
| | Pentadecanoic acid | 0.896 | 89.3% | 89.9% |
| Taurine | Arachidonic acid | 0.892 | 88.8% | 89.4% |
| | Citramalic acid | 0.889 | 87.6% | 89.1% |
| | Methionine | 0.884 | 87.1% | 87.9% |
| | Pentadecanoic acid | 0.881 | 86.4% | 87.1% |
| Arachidonic acid | Citramalic acid | 0.879 | 86.7% | 86.5% |
| | Methionine | 0.869 | 85.5% | 86.3% |
| | Pentadecanoic acid | 0.865 | 84.8% | 86.0% |
| Citramalic acid | Methionine | 0.848 | 83.1% | 85.0% |
| | Pentadecanoic acid | 0.842 | 82.7% | 83.4% |
| Methionine | Pentadecanoic acid | 0.831 | 80.8% | 81.6% |

TABLE 3
Combination of any three to five of the 6 differential
metabolites to distinguish SA from ACS

| Combined numbers | AUC | Sensitivity | Specificity |
|---|---|---|---|
| Three | ≧0.921 | ≧92.2% | ≧91.6% |
| Four | ≧0.933 | ≧92.7% | ≧93.3% |
| Five | ≧0.937 | ≧95.0% | ≧94.8% |

Table 1 shows that single use of the 6 differential metabolites provided clinically diagnostic value of SA vs. ACS with AUC>0.7. Table 2 shows that any two of the six differential metabolites provided high AUC with high sensitivity and specificity with potential clinically diagnostic value. Table 3 shows that any three to five metabolites of the six differential metabolites provided higher AUC than any two of the six differential metabolites with potential clinically diagnostic value.

Therefore, the six differential metabolites can be used as metabolic biomarkers for differential diagnosis of SA vs. ACS.

### Embodiment 3

### Preparation of Detection Kit.

Detection kits were prepared on the basis of the metabolic biomarkers provided by the invention. The detection kit includes the following constituent.

Reference standards of the metabolic biomarkers are included. They are malic acid, taurine, arachidonic acid, citramalic acid, methionine and pentadecanoic acid. They are individually packaged.

Solvents to extract the metabolic biomarkers from plasma are included. They are pure acetonitrile and 20% acetonitrile water solution for sample preparation in UPLC-Q/TOF-MS analysis, mixed solution of methanol, chloroform, water (2.5:1:1, V/V/V), methoxamine pyridine solution and N-methyl-N-(trimethylsilyl)trifluoroacetamide for sample preparation in GC-Q/MS analysis. In UPLC-Q/TOF-MS analysis, 20% acetonitrile water solution can be used as the solvent to dissolve the reference standards. In GC-Q/MS analysis, the standard solutions are prepared as sample preparation.

Internal standard citramalic acid is included.

Of course, not all the six metabolic biomarkers are required. The detection kit may include some of them. The reference standards are individually packaged or packaged together.

The detection kit is designed based on the metabolic biomarkers published by the invention, so it can be applied to distinguish SA from ACS.

In summary, the invention effectively overcomes the disadvantages in the existing technology with high industrial potential.

The above examples discuss the essentials of the invention. The protection scope of the invention is not limited to the examples. Replacement, modifications and changes are necessary and straight forward to researchers in this area, and thus should be part of this invention.

1-10. (canceled)

11. A method for differentiating between subjects with stable angina pectoris (SA) and subjects with acute coronary syndrome (ACS), the method comprising the steps of:
obtaining a plasma sample from the subject;
extracting one or more metabolic biomarkers from the plasma sample using acetonitrile as an extraction solvent, wherein the one or more metabolic biomarkers are selected from the group consisting of malic acid, taurine, citramalic acid, methionine, and pentadecanoic acid;
determining the levels of the extracted one or more metabolic biomarkers in the plasma sample; and
comparing the levels of the said one or more metabolic biomarkers in the subject with predetermined reference values resulting in a differentiating between subjects with SA and subjects with ACS, wherein the levels of the said metabolic biomarkers are different between subjects with SA and subjects with ACS;
wherein the steps of extracting the one or more metabolic biomarkers and determining the levels of said one or more metabolic biomarkers is done by LC-MS and/or GC-MS comprising the steps of:
wherein profiling by UPLC-Q/TOF-MS comprises the following steps:
applying response surface method for optimizing the extraction solvent;
evaluating the data by using multivariate analysis;
determining the importance of variables to model response;
using acetonitrile as extraction solvent to extract plasma samples;
adding acetonitrile into plasma sample, mixing about 30 seconds by vortex,
centrifugating about 10 minutes at about 13,000 rotation/min at about 4 centigrade,
transferring supernatant to another centrifuge tube,
drying the liquid with nitrogen in room temperature,
using acetonitrile water solution to dissolve the residue,
obtaining a plurality of first parameters by using chromatography,
wherein profiling by GC-Q/MS comprises the following steps;
placing plasma into another centrifuge tube,
adding citramalic acid solution, mixed solution of methanol, chloroform and water Into the centrifuge tube,
shaking the centrifuge tube for about 30 minutes at about 1200 rpm at constant temperature,
centrifuging for about 5 minutes at about 16,000 g at about 4 centigrade,
transferring supernatant to another centrifuge tube,
drying the liquid with nitrogen at room temperature,
adding methoxamine pyridine to dissolve the residue,
oximating for about 8 hours at about 50 centigrade degrees,
adding N-methyl-N-trifluoroacetamide and reacted about 2 hours at about 70 degree centigrade,
obtaining a plurality of second parameters by using chromatography
analyzing the plurality of first parameters and the plurality of second parameters by screening six differential metabolites including malic acid, taurine, arachidonic acid, citramalic acid, methionine, pentadecanoic acid,
using the plurality of first parameters and the plurality of second parameters to determine for differential diagnosis of SA and ACS.

12. The method for differentiating between subjects with SA and subjects with ACS according to claim 11, wherein two metabolic biomarkers are extracted from the plasma sample.

13. The method for differentiating between subjects with SA and subjects with ACS according to claim 11, wherein three metabolic biomarkers are extracted from the plasma sample.

14. The method for differentiating between subjects with SA and subjects with ACS according to claim 11, wherein four metabolic biomarkers are extracted from the plasma sample.

15. The method for differentiating between subjects with SA and subjects with ACS according to claim 11, wherein five metabolic biomarkers are extracted from the plasma sample.

16. The method for differentiating between subjects with SA and subjects with ACS according to claims 11, wherein the extracted metabolic biomarkers further comprise arachidonic acid.

17. A method for determining whether SA has developed to ACS in a subject, the method comprising the steps of: obtaining a plasma sample from the subject;
extracting six specific metabolic biomarkers from the plasma sample using acetonitrile as an extraction solvent, wherein the six specific metabolic biomarkers are malic acid, taurine, arachidonic acid, citramalic acid, methionine, and pentadecanoic acid;
determining the levels of the six specific metabolic biomarkers in the plasma sample; and
comparing present levels of the said metabolic biomarkers in the subject with previous average levels of the said metabolic biomarkers in the subject, wherein 0.7-0.8 times down-regulation of malic acid and citramalic acid and 0.7-0.8 times up-regulation of taurine, arachidonic acid, methionine and pentadecanoic acid indicate SA has developed to ACS in the subject;
wherein the step of extracting the six specific metabolic biomarkers and determining the levels of the six specific metabolic biomarkers is done by LC-MS and/or GC-MS.
